(19) United States
(12) Patent Application Publication
(10) Pub. No.: US 2010/0222825 A1
(43) Pub. Date: Sep. 2, 2010

(54) SIDE-LOADING OCCIPITAL VERTEBRAL FIXATION SYSTEM

(75) Inventors: Marc PAUL, Memphis, TN (US); Ben Cowan, Memphis, TN (US); Jayant Jangra, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/396,335

(22) Filed: Mar. 2, 2009

(51) Int. Cl. A61B 17/70

(52) U.S. Cl. 606/280; 606/250

(57) ABSTRACT

An occipital plating system includes a fixation assembly with a plate and a coupling member extending from a respective one of opposite lateral portions of the plate. The coupling members each include a passage that opens in at least one of a medial or lateral direction to receive a connecting element extending from the spinal column.

### BACKGROUND

Various devices have been employed for fixation of the occiput with the cervical vertebrae. Posterior systems include a plate attached to the occiput with screw fixation. A rod extends from the plate and along the cervical vertebrae for attachment to the cervical vertebrae to provide a platform for fixation. Spanning of single or multiple levels of the cervical spine results in fixation of these levels in conjunction with the occiput.

In the cervical region, anatomical considerations can make it difficult to provide a plate that adequately fits in the space along the occiput. Furthermore, securing rods or other connecting elements to the occipital plate can be difficult. Systems for occipital and cervical stabilization are needed that provide adequate stabilization while providing an optimal fit with the occiput and vertebral anatomy. Such systems should further reduce the invasiveness and complexity of the procedure.

### SUMMARY

An occipital plating system includes a plate with a profile adapted for positioning on the occiput. The plate extends bi-laterally across the central axis of the spinal column to opposite lateral portions of the plate. At least one coupling member extends from each of the lateral portions of the plate and includes a medially or laterally oriented opening to receive a connecting element extending from the spinal column so the proximal surface of the coupling member presents a smooth surface profile in the proximal direction. The orientation and configuration of the coupling members also provide a lower profile for the plate in the proximal direction. The coupling members eliminate the need for openings, set screws, clamps or other connecting element securing or receiving structures that engage or receive the connecting element to the coupling member from a proximally oriented direction Patient comfort is increased by eliminating sharp or abrupt corners or changes in surface profiles in the coupling members in areas where the coupling members are directed toward the skin of the patient when the plate is implanted.

According to one aspect, an occipital plating system includes a plate with a central portion extending along a medial-lateral axis between opposite lateral portions. The plate further includes a top surface facing a proximal direction and a bottom surface opposite the top surface so that the bottom surface faces distally toward an occiput. The top and bottom surfaces define a first, substantially constant thickness along the central and lateral portions. The central portion of the plate includes at least one fastener hole extending between the top and bottom surfaces to receive a fastener therethrough. The lateral portions are located on opposite sides of a central axis of the plate and each of the lateral portions includes a coupling member associated therewith projecting proximally from the top surface of the central portion. The coupling members each are configured to engage a respective connecting element extending from a vertebra to the occiput. Each of the coupling members extends from the associated lateral portion along the medial-lateral axis and includes a passage extending therethrough that generally parallels the central axis for receiving the respective connecting element through the coupling member. Each of the coupling members also defines an internal bore opening into the passage. The internal bore extends from the passage along the medial-lateral axis and opens at one of a medial side and a lateral side of the coupling member. Each of the coupling members also includes a portion of the top surface defining a variably thickness with the bottom surface, and the portion of the top surface is convexly curved around the medial-lateral axis from the bottom surface toward where the top surface defines the first thickness of the plate. The convexly curved surface extends between the medial side and the lateral side of the coupling member so that the internal bore and the passage do not open in the proximal direction.

According to another aspect, an occipital plating system above include first and second coupling members associated with and projecting from respective lateral portions of a plate extending from opposite sides of a central portion of the plate. The plate further includes a top surface facing a proximal direction and a bottom surface opposite the top surface where the bottom surface is for positioning distally toward an occiput, and the central portion of the plate includes at least one fastener hole extending between the top and bottom surfaces to receive a fastener therethrough. Each of the coupling members extends from its associated lateral portion along the medial-lateral axis and includes a passage extending therethrough that generally parallels the central axis for receiving the respective connecting element through the coupling member. Each of the coupling members further defines an internal bore opening into the passage with the internal bore extending from the passage along the medial-lateral axis and opening at one of a medial side and a lateral side of the coupling member. Each of the coupling members further includes a portion of the top surface defining a variable thickness with the bottom surface and the portion of the top surface is substantially uninterrupted in a proximally facing direction of the plate from the medial side to the lateral side of the coupling member with the passage opening along cephaladly and caudally facing sides of the coupling member. The portion of the top surface is convexly curved between the openings of the passage at the cephaladly and caudally facing sides of the coupling member.

These and other aspects will also be apparent from the following description and the Figures.

### BRIEF DESCRIPTION OF THE DRAWINGS

FIG. 1 shows an occipital vertebral fixation system looking distally toward the occiput.

FIG. 2 shows a perspective view of a fixation assembly comprising a portion of the system of FIG. 1.

FIG. 3 shows a perspective view of another embodiment fixation assembly of the system of FIG. 1.

### DESCRIPTION OF THE ILLUSTRATED EMBODIMENTS

For the purpose of promoting an understanding of the principles of the invention, reference will now be made to the illustrated embodiments thereof and specific language will be used to describe the same. It will nevertheless be understood that no limitation of the scope of the invention is thereby intended. Any such alterations and further modifications in the invention, and any such further applications of the principles of the invention as described herein are contemplated as would normally occur to one skilled in the art to which the invention relates.

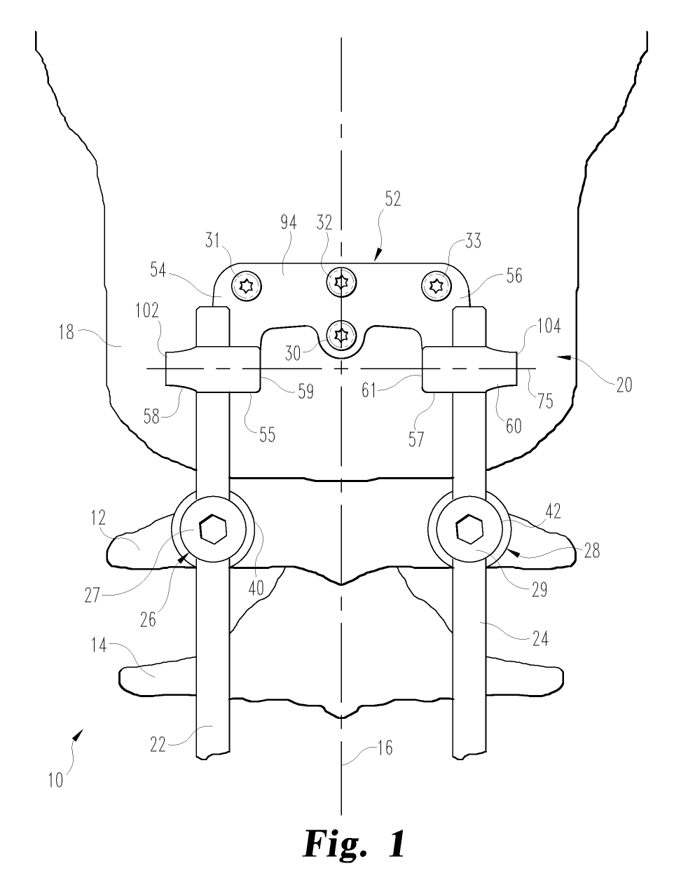

Referring to FIG. 1, there is shown a posterior elevation view of a spinal column segment 10 including the upper cervical vertebra C1 designated at 12 and the next lower cervical vertebra C2 designated at 14. Cervical vertebrae 12, 14 extend along the central axis 16 of spinal column segment 10. Occiput 18 resides at the superior end of C1 vertebra 12. Occipital fixation system 20 includes an occiput fixation assembly 50 engaged to occiput 18 and a pair of elongated connecting elements 22, 24 engaged to at least one of the cervical vertebrae 12, 14. Connecting elements 22, 24 are bi-laterally positioned along opposite sides of the spinous processes of one or more of the cervical vertebrae and central axis 16 of the spinal column, and engaged to posterior spinal elements on one or more cervical vertebrae with one or more anchors 26, 28, respectively. One or both of connecting elements 22, 24 may further extend along the spinal column to one or more of the thoracic, lumbar and sacral regions and engaged to one or more vertebrae thereof with any suitable connector.

Connecting elements 22, 24 extend along the spinal column and can be rigid to prevent motion, flexible, or partially flexible to allow at least limited motion in the stabilized motion segments. The connecting elements can be in the form of rods, plates, bars, tethers, cables, dampeners, springs, or other structure to provide a desired stabilization effect. The connecting elements 22, 24 can be secured to one or more of the vertebrae with any suitable bone anchoring device or devices. In the illustrated embodiment, bone anchors 26, 28 are engaged to vertebra 12 and also to connecting elements 22, 24 with engaging members 27, 29 engaged to the respective bone anchors 26, 28.

The bone anchors 26, 28 can include bone screws with a receiving portion 40, 42, respectively, for receiving the respective connecting element 22, 24. The receiving portion 40, 42 can be movable relative to the bone engaging portion or fixed relative to the bone engaging portion of the respective anchoring device 26, 28. The receiving portions 40, 42 can be in the form of a top-loading, side-loading or bottom loading member with a channel to receive the respective connecting element 22, 24. Engaging members 27, 29 can be provided in the form of a set screw, nut, cap, slide-lock device, or other device that can be engaged to the respective receiving portion 40, 42 to secure the connecting element 22, 24 in the respective receiving portion 40, 42. The receiving portions 40, 42 can also be in the form of a clamp, wire or other device positioned around the connecting element 22, 24 to secure it to the bone anchoring portion. The bone anchoring portion (not shown) can be in the form of a bone screw, hook, cable, anchor, staple, interbody device, clamp, or other suitable bone engaging structure to secure the respective connecting element 22, 24 to the corresponding bony structure.

Fixation assembly 50 secures at least one of the connecting elements 22, 24 to the occiput 18. Fixation assembly 50 includes a plate 52 engaged to the occiput with one or more fasteners 30, 31, 32 and 33. Fasteners 30, 31, 32 and 33 can be in the form of bone screws with a threaded shaft (not shown) and a proximal head (shown) that is positioned against a proximally oriented surface of plate 52 to secure it against or adjacent to the occiput. In the illustrated embodiment, four fasteners are shown and plate 52 includes four holes to accommodate respective ones of the four fasteners. It is contemplated that fewer than four fasteners may be provided so that one or more holes do not include a fastener. Furthermore, plate 52 may be provided with fewer than four holes or more than four holes, and fasteners can be provided and inserted through all or a portion of the holes provided in plate 52.

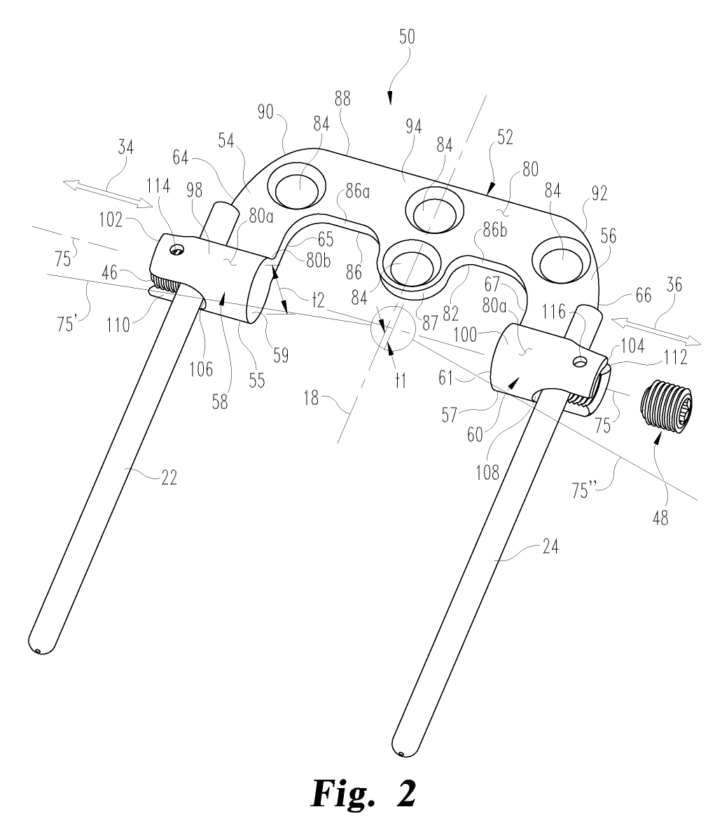

Referring farther to FIG. 2, plate 52 is elongated in a direction extending along medial-lateral axis 75, and is symmetrical about a central axis 18 of plate 52. When secured to the occiput, plate 52 includes a central portion 94 that extends transversely to the central axis 16 of the spinal column in a direction along medial-lateral axis 75 to opposite lateral portions 54, 56, and central axis 18 of plate 52 is positioned on or generally parallel to central axis 16. Lateral portions 54, 56 are located bi-laterally relative to central axis 16 and offset caudally from central portion 94 to laterally offset coupling member 58, 60 to opposite sides of the medial nuchal line and caudally from the respective lateral portion 54, 56. The laterally and caudally located outer ends 55, 57 each include a respective one of coupling members 58, 60 formed as a unitary, integral structure with lateral portions 54, 56 and central portion 94. Coupling members 58, 60 extend proximally away from the occiput from the respective lateral portion 54, 56 to receive the respective connecting element 22, 24 therein. An engaging member 46, 48 such as a set screw, cap, nut or other device can be engaged to each of the coupling members 58, 60 to secure the respective connecting element 22, 24 to, in, on or around coupling members 58, 60. Alternatively, coupling members 58, 60 can be configured to crimp, clamp, frictionally lock, capture or otherwise secure the respective connecting element 22, 24 thereto without a separate engaging member.

Fixation assembly 50 is configured so that coupling members 58, 60 are arranged to receive the respective connecting element 22, 24 therein from a direction that is along, generally parallel to, or slightly obliquely oriented to medial-lateral axis 75, as indicated by arrows 34, 36, to provide a desired fit and location of coupling members 58, 60 relative to the occipital and cervical anatomy and the connecting elements 22, 24. For example, connecting element 22 can be moved medially toward central axes 16, 18 and along medial-lateral axis 75 relative to lateral portion 54 and coupling member 58 for placement into the laterally opening coupling member 58, as indicated by bi-directional arrow 34, and connecting element 24 can be moved medially toward central axes 16, 18 and along medial-lateral axis 75 relative to lateral portion 56 and coupling member 60, as indicated by bi-directional arrow 36, for placement into the laterally opening coupling member 60. In another example, coupling members 58, 60 extend along or parallel to axes 75′, 75″, respectively, that are slightly obliquely oriented to medial-lateral axis 75 so that openings into coupling members 58, 60 that receive connecting elements 22, 24 are oriented more caudally, further reducing the lateral profile of fixation assembly 50 and orienting the openings into coupling members 58, 60 in a direction to readily accept connecting elements 22, 24 extending cephaladly from the vertebrae. Accordingly, in this example connecting elements 22, 24 are moved medially toward central axes 16, 18 and along axes 75′, 75″ relative to lateral portions 54, 56 and coupling members 58, 60 for placement into the laterally opening coupling members 58, 60, as indicated by bi-directional arrows 34, 36. The lateral-medially directed placement of connecting elements 22, 24 into the respective coupling members 58, 60 minimizes the profile of plate 52 extending from occiput 18, and allows plate 52 to present a smooth surface profile along its proximally directed facing surfaces since no proximally outwardly directed openings or other proximal surface interruptions are required to receive the connecting elements or engaging members that secure the connecting elements 22, 24 to coupling members 58, 60.

Plate 52 includes central portion 94 with a top surface 80 and a bottom surface 82 opposite top surface 80. Bottom surface 82 is positioned or oriented distally against or adjacent the bone of the occiput when plate 52 is implanted in the patient. Holes 84 extend between and open at top and bottom surfaces 80, 82. Plate 52 also includes a caudally oriented sidewall 86 extending between top and bottom surfaces 80, 82 and an opposite cephaladly oriented sidewall 88 extending between top and bottom surfaces 80, 82. Caudal sidewall 86 includes a convexly curved central lobe 87 centered on central axis 18 that forms a convexly curved sidewall portion across central axis 18. Caudal sidewall 86 also includes linear sidewall portions 86a, 86b between central lobe 87 and medial walls 65, 67 of lateral portions 54, 56, respectively. Medial walls 65, 67 parallel central axis 18 and extend to the respective end 55, 57 of lateral portions 54, 56. Cephalad sidewall 88 is substantially linear across central axis 18 between lateral portions 54, 56. Lateral portions 54, 56 each include a convexly curved sidewall 90, 92, respectively, that extends from cephalad sidewall 88 to respective ones of the lateral most sidewalls 64, 66 of lateral portions 54, 56. The central lobe 87 between lateral portions 54, 56 provides plate 52 with an E-shaped configuration with the opening of the E shape oriented in the caudal direction when plate 52 implanted and secured to the occiput.

Holes 84 are distributed about central portion 94 with three holes 84 located adjacent to and following cephalad sidewall 88 and one hole 84 located in lobe 87. Holes 84 provide multiple locations and options for surgeon placement of fasteners to secure plate 52 to the occiput, while the E-shaped plate 52 and hole arrangement minimize the area of the occiput occupied by plate 52. Furthermore, holes 84 and recessed areas formed on the sides of lobe 87 with lateral portions 54, 56 provide plate 52 with flexibility so that central portion 94 can be bent and flexed to conform to the patient's anatomy when implanted, further reducing the profile of plate 52 extending proximally from the occiput. Other hole arrangements for plate 52 are not precluded.

Fixation assembly 50 may be provided with a retaining system (not shown) to prevent one or more of the fasteners in holes 84 from backing out relative to plate 52. A retaining system can be provided for each bone engaging fastener, or a single retaining system can be provided for all bone engaging fasteners. Other suitable retaining systems are contemplated, including lock washers, caps and devices that expand the heads of the bone engaging fasteners, or plates, washers or set screws that cover or overlap the heads of one or more of the bone fasteners in holes 84. It is further contemplated that fixation assembly 50 can be provided without any retaining systems.

Central portion 94 of plate 52 extends between lateral portions 54, 56, which extend from central portion 94 to respective ones of the coupling members 58, 60. Central portion 94 and lateral portions 54, 56 include a constant first thickness t1 between top and bottom surfaces 80, 82. In other embodiments, first thickness t1 is substantially constant across central portion 94 and lateral portions 54, 56. Bottom surface 82 extends along coupling members 58, 60 so that coupling members 58, 60 and the associated lateral portions 54, 56 are co-planar along bottom surface 82. Coupling members 58, 60 include top surface portion 80a that varies in distance from bottom surface 82 along coupling members 58, 60. Coupling members 58, 60 define a second thickness t2 between top surface portion 80a and bottom surface 82. Thickness t2 varies depending on the location of the coupling members 58, 60 where the thickness of coupling members 58, 60 is measured. In the illustrated embodiment, the variable thickness t2 is formed by the portion 80a of top surface 80 being convexly rounded around medial-lateral axis 75 from bottom surface 82 toward top surface 80 along the associated lateral portion 54, 56. Top surface portion 80a projects outwardly or proximally from bottom surface 82 from top surface 80 to provide coupling members 58, 60 with a raised, proximally extending profile relative to top surface 80 along central portion 94 and lateral portions 54, 56. Top surface portion 80a forms a concavely curved transition 80b extending from the convexly curved portion of top surface portion 80a to where it joins top surface 80 at the associated lateral portion 54, 56.

In the illustrated embodiment of FIG. 2, coupling members 58, 60 are identical to one another and arranged symmetrically about central axis 18 of plate 52. Coupling member 58 includes a blind end 59 aligned with medial wall 65 of lateral portion 54. Coupling member 58 includes a body 98 that extends along medial-lateral axis 75, or along axis 75′, from blind end 59 to lateral end 102. Blind end 59 is aligned with medial wall 65 of lateral portion 54, and lateral end 102 projects laterally from lateral wall 64 of lateral portion 54. Body 98 defines a U-shaped passage 106 that extends in a direction paralleling or generally parallel to central axis 18 to open at the caudal and cephalad sides of coupling member 58 to allow connecting element 22 to extend through coupling member 58 in a direction generally paralleling central axis 18. Coupling member 58 further includes a bore 110 opening at lateral end 102 and extending into passage 106. Body 98 defines internal threads therealong for threadingly engaging the corresponding engaging member 46. Engaging member 46 is threaded medially along medial-lateral axis 75, or along axis 75′ for embodiments oriented therealong, and into bore 110 toward central axis 18 to secure connecting element 22 in passage 106. Bore 110 and passage 106 are oriented so that neither extends to or opens at or along the proximally facing portion of top surface portion 80a, and the convexly curved top surface portion 80a extends between the cephaladly and caudally opening sides of passage 106 to providing a smooth profile for coupling member 58 along its proximally directed surface that faces toward the skin of the patient after the surgery is complete. In the illustrated embodiment, a small hole 114 is provided in top surface portion 80a to receive an instrument to assist in positioning plate 52 into the patient. Other embodiments contemplate that hole 114 is eliminated.

Coupling member 60 includes a blind end 61 aligned with medial wall 67 of lateral portion 56. Coupling member 60 includes a body 100 that extends from blind end 61 to lateral end 104 along medial-lateral axis 75, or along axis 75″ for embodiments oriented therealong. Blind end 61 is aligned with medial wall 67 of lateral portion 56, and lateral end 104 projects laterally from lateral wall 66 of lateral portion 56. Body 100 defines a U-shaped passage 108 that extends in a direction paralleling central axis 18 and opening at the caudal and cephalad sides of coupling member 60 to allow connecting element 24 to extend through coupling member 60 in a direction generally paralleling central axis 18. Coupling member 60 further includes a bore 112 opening at lateral end 104 and extending into passage 108. Body 100 defines internal threads therealong for threadingly engaging the corresponding engaging member 48. Engaging member 48 is threaded medially along medial-lateral axis 75, or along axis 75″, into bore 112 toward central axis 18 to secure connecting element 24 in passage 108. Bore 112 and passage 108 are oriented so that neither extends to or opens at or along the proximally directed portion of top surface portion 80a, and the convexly curved top surface portion 80a extends between the cephaladly and caudally opening sides of passage 108 to providing a smooth profile for coupling member 60 along its proximally directed surface that faces toward the skin of the patient after the surgery is complete. In the illustrated embodiment, a small hole 116 is provided in top surface 80a to receive an instrument to assist in positioning plate 52 into the patient. Other embodiments contemplate that hole 116 is eliminated.

When plate 52 is secured to the occiput with central axis 18 aligned on or parallel to central axis 16, connecting elements 22, 24 are moved medially toward central axis 18 for placement into the respective coupling member 58, 60. The proximal side of coupling members 58, 60 is enclosed with the respective body 98, 100 to prevent any placement or withdrawal of connecting elements 22, 24 from coupling members 58, 60 in the proximal direction. Engaging members 46, 48 are then engaged to the respective coupling members 58, 60 from this same lateral toward medial approach.

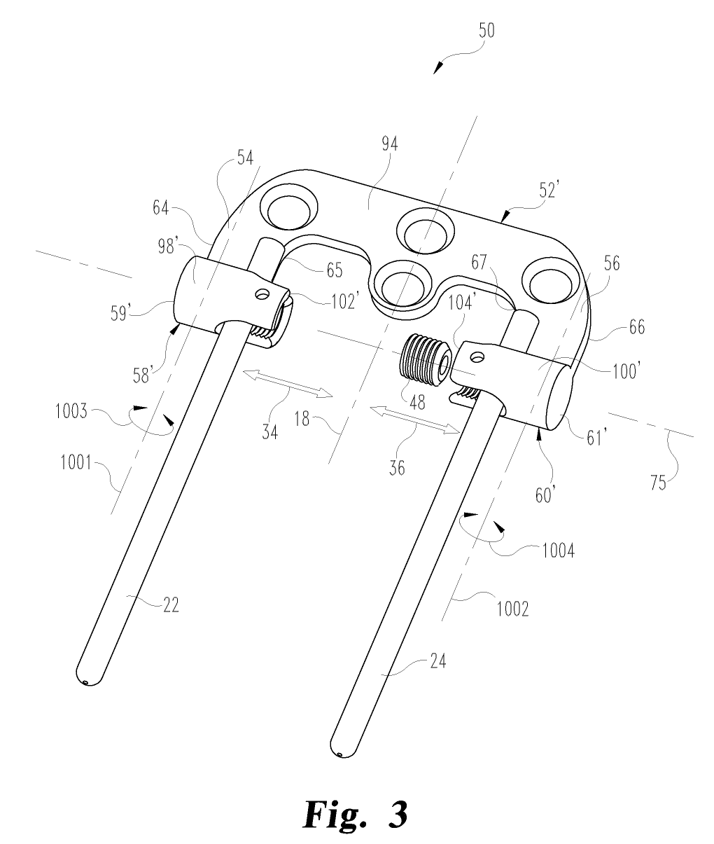

Another embodiment plate 52′ is provided with coupling members 58′, 60′ that are oriented so that each opens medially, as shown in FIG. 3. Plate 52′ is identical to plate 52 discussed herein except for the orientation of the coupling members 58′, 60′ and location of coupling members 58′, 60′ relative to lateral portions 54, 56. In FIG. 3, the lateral most side of coupling member 58′ is formed by blind end 59′ of coupling member 58′, and blind end 59′ is aligned with the lateral most side 64 of lateral portion 54. For coupling member 60′, the lateral most side is formed by blind end 61′ which is aligned with the lateral most side 66 of lateral portion 56. The body 98′ of coupling member 58′ extends from blind end 59′ across end 55 and projects medially from medial side 65 of lateral portion 54 to a medial end 102′. The body 100′ of coupling member 60′ extends from blind end 61′ across end 57 and projects medially from medial side 67 of lateral portion 54 to a medial end 104′.

For plate 52′, connecting elements 22, 24 are moved laterally away from central axis 18 and along or in the general direction of medial lateral axis 75 for placement into the respective coupling member 58′, 60′. Engaging members 46, 48 are then engaged to the respective coupling members 58′, 60′ from this same medial toward lateral approach away from central axis 18. The embodiment of FIG. 3 minimizes the lateral exposure of the occipital area required to accommodate placement of connecting elements 22, 24 into coupling members 58′, 60′. In other embodiments, a plate is provided with one of the coupling members 58, 60 oriented to open laterally while the other of the coupling members 58′, 60′ is oriented to open medially.

One technique for implanting the system 20 will be described. Plate 52, 52′ can be contoured to lie along and against the occiput. Plate 52, 52′ can be placed along the median nuchal line of the occiput so that coupling members 58, 60 or coupling members 58′, 60′ lie either along or lateral to the median nuchal line and bi-laterally relative to the central axis of the spinal column. Connecting elements 22, 24 can be positioned for engagement to coupling members 58, 60 or coupling members 58′, 60′ by the surgeon during surgery and engaged thereto with, for example, engaging members 46, 48 when the desired fit for system 20 has been obtained. Connecting elements 22, 24 can be provided in straight or pre-bent configurations, and in either configuration can be positioned without further bending, or can be further bent, for engagement to coupling members 58, 60 or coupling members 58′, 60′. Connecting elements 22, 24 can be pre-contoured to fit the patient's anatomy extending along the spinal column, or contoured during surgery to provide a desired custom fit with the patient.

Fixation assembly 50 is provided with occipital plate 52, 52′ that includes a low profile both extending proximally or away from the occiput and also extending medially-laterally relative to the occiput. Placement and securing of one or more fusion devices and/or fusion material in the disc space or other location between one or more of the vertebrae along which connecting elements 22, 24 extend and its adjacent vertebrae are also contemplated. Further stabilization of one or more of the cervical vertebrae with one or more anterior plating systems is also contemplated in addition to the fixation assembly 50.

In the embodiments illustrated above, central axis 18 and medial-lateral axis 75 intersect one another and lie in a common plane extending in the cephalad and caudal directions. It is contemplated that in one embodiment coupling members 58, 60 or coupling member 58′, 60′ are arranged so that the connecting elements 24 and the respective engaging members 46, 48 are moved medially toward central axes 16, 18 or laterally away from central axes 16, 18 in the common plane or in a plane parallel to or generally parallel to the common plane defined by central axis 18 and medial-lateral axis 75 for placement into the corresponding coupling member. For embodiments where coupling members 58, 60 or coupling members 58′, 60′ are oriented along oblique axes 75′, 75″, connecting elements 24 and the respective engaging members 46, 48 are moved medially toward central axes 16, 18 or laterally away from central axes 16, 18 along the oblique axes 75′, 75″ and in the common plane or in a plane parallel to or generally parallel to the common plane defined by central axis 18 and medial-lateral axis 75 for placement into the corresponding coupling member. In still other embodiments, coupling members 58, 60 or coupling members 58′, 60′ extend anteriorly or posteriorly in an oblique orientation to the common plane so that the connecting elements 24 and the respective engaging members 46, 48 are moved medially toward central axes 16, 18 or laterally away from central axes 16, 18 in the oblique orientation to the common plane defined by central axis 18 and medial-lateral axis 75 for placement into the corresponding coupling member.

With reference now to FIG. 3, there are shown offset axes 1001, 1002 extending through coupling members 58′, 60′ adjacent to blind ends 59′, 61′. Offset axes 1001, 1002 are parallel to central axis 18 and orthogonal to medial-lateral axis 75. In FIG. 3, coupling members 58′, 60′ each extend from its respective offset axis 1001, 1002 in a reference direction that forms an azimuth of 0 degrees. In FIG. 2, each of the coupling members 58, 60 is rotated around its respective offset axis 1001, 1002, as indicated by arrows 1003, 1004, so that each coupling member 58, 60 is oriented at an azimuth of 180 degrees with respect to reference direction established by the corresponding coupling member 58′, 60′ of FIG. 3. Other embodiments contemplate that the coupling members extend from the respective axis 1001, 1002 at any azimuth from the reference direction other than 90 degrees and 270 degrees with respect to the offset axis 1001, 1002. These orientations of the coupling members that are intermediate those shown in FIGS. 2 and 3 may be advantageous in insertion of engaging members into the coupling members while still providing a reduced profile.

While the invention has been illustrated and described in detail in the drawings and foregoing description, the same is to be considered as illustrative and not restrictive in character. All changes and modifications that come within the spirit of the invention are desired to be protected. For example, one of coupling members 58, 60 may be fixed relative to its respective lateral portion 54, 56, or movable relative to all or a portion of plate 52 by another engagement relationship with plate 52. Also, other shapes for central portion 94 and lateral portions 54, 56 are contemplated.

What is claimed is:

1. An occipital plating system, comprising:
a plate including a central portion extending along a medial-lateral axis between opposite lateral portions, said plate further including a top surface facing a proximal direction and a bottom surface opposite said top surface, said bottom surface for positioning distally toward an occiput, said top and bottom surfaces defining a first thickness along said central and lateral portions, said central portion of said plate including at least one fastener hole extending between said top and bottom surfaces to receive a fastener therethrough, said lateral portions being located on opposite sides of a central axis of said plate and each of said lateral portions includes a coupling member associated therewith projecting proximally from said top surface of said central portion, said coupling members each being configured to engage a respective connecting element extending from a vertebra to the occiput, wherein each of said coupling members:
extends from said associated lateral portion generally along said medial-lateral axis and includes a passage extending therethrough that generally parallels said central axis for receiving the respective connecting element through said coupling member;
defines a bore opening into said passage, said bore extending from said passage generally along said medial-lateral axis and opening at one of a medial side and a lateral side of said coupling member; and
includes a top surface portion that defines a variable thickness with said bottom surface, said top surface portion is convexly curved around said medial-lateral axis from said bottom surface toward where said top surface defines said first thickness with said bottom surface, said convexly rounded surface extending from said medial side to said lateral side of said coupling member so that said internal bore and said passage do not open in said proximal direction.

2. The plating system of claim 1, wherein said plate includes:
a first sidewall extending linearly across said central axis at a cephalad end of said plate, said first sidewall further extends between said top and bottom surfaces of said plate; and
a second sidewall extending across said central axis at a caudal end of said plate between said lateral portions, wherein said second sidewall extends around a lobe centered on and extending caudally from said central portion along said central axis, and said second sidewall further extends between said top and bottom surfaces of said plate.

3. The plating system of claim 2, wherein said at least one hole includes three holes through said central portion adjacent to said first sidewall, and said at least one hole further includes a single hole through said lobe.

4. The plating system of claim 1, wherein said top surface is concavely curved from each of said coupling member to said central portion.

5. The plating system of claim 1, wherein said bore is threaded and further comprising first and second engaging members threadingly engaged to said respective ones of said coupling members in said bore.

6. The plating system of claim 1, wherein for each of said coupling members said bore opens at said lateral side of said coupling member and each of said coupling members includes a blind end adjacent to said central axis opposite said bore opening.

7. The plating system of claim 6, wherein said lateral portions of said plate each include a lateral wall and a medial wall each paralleling said central axis, said medial wall being located more adjacent to said central axis than said lateral wall, each of said coupling members projecting laterally from said lateral wall of said respective lateral portion to said lateral side of said coupling member.

8. The plating system of claim 1, wherein for each of said coupling members said bore opens at said medial side of said coupling member and each of said coupling members includes a blind end adjacent to said central axis and opposite said bore opening.

9. The plating system of claim 8, wherein said lateral portions of said plate each include a lateral wall and a medial wall each paralleling said central axis, said medial wall being located more adjacent to said central axis than said lateral wall, each of said coupling members projecting medially from said medial wall of said respective lateral portion to said medial side of said coupling member.

10. The plating system of claim 1, wherein said coupling members and said central and lateral portions of said plate form a unitary construct.

11. An occipital plating system, comprising:
a plate including a central portion extending along a medial-lateral axis between opposite lateral portions, said plate further including a top surface facing a proximal direction and a bottom surface opposite said top surface, said bottom surface for positioning distally toward an occiput, said top and bottom surfaces defining a first thickness along said central and lateral portions, said central portion of said plate including at least one fastener hole extending between said top and bottom surfaces to receive a fastener therethrough, said lateral portions being located on opposite sides of a central axis of said plate and each of said lateral portions includes a coupling member associated therewith projecting proximally from said top surface along said lateral and central portions, said coupling members each being configured to engage a respective connecting element extending from a vertebra to the occiput, wherein each of said coupling members:
extends from said associated lateral portion along said medial-lateral axis and includes a passage extending therethrough that generally parallels said central axis and opens at cephaladly and caudally facing sides of said coupling member for receiving the respective connecting element through said coupling member;
defines an internal bore opening into said passage, said internal bore extending from said passage along said medial-lateral axis and opening at one of a medial side and a lateral side of said coupling member; and
includes a top surface portion defining a variable thickness with said bottom surface, wherein said top surface portion is substantially uninterrupted in a proximally facing direction of said plate from said medial side to said lateral side of said coupling member and said top surface portion is convexly curved between said openings of said passage at said cephaladly and caudally facing sides of said coupling member.

12. The plating system of claim 11, wherein for each of said coupling members said bore opens at said lateral side of said coupling member and each of said coupling members includes a blind end adjacent said central axis opposite said bore opening.

13. The plating system of claim 12, wherein said lateral portions of said plate each include a lateral wall and a medial wall each paralleling said central axis, said medial wall being located more adjacent to said central axis than said lateral wall, each of said coupling members projecting laterally from said lateral wall of said respective lateral portion.

14. The plating system of claim 13, wherein said plate includes:
a first sidewall extending linearly across said central axis at a cephalad end of said plate, said first sidewall further extends between said top and bottom surfaces of said plate; and
a second sidewall extending across said central axis at a caudal end of said plate between said lateral portions, wherein said second sidewall extends around a lobe centered on and extending caudally from said central portion along said central axis, and said second sidewall further extends between said top and bottom surfaces of said plate.

15. The plating system of claim 11, wherein for each of said coupling members said bore opens at said medial side of said coupling member and each of said coupling members includes a blind end adjacent to said central axis and opposite said bore opening.

16. The plating system of claim 15, wherein said lateral portions of said plate each include a lateral wall and a medial wall each paralleling said central axis, said medial wall being located more adjacent to said central axis than said lateral wall, each of said coupling members projecting medially from said medial wall of said respective lateral portion to said medial side of said coupling member.

17. The plating system of claim 16, wherein said plate includes:
a first sidewall extending linearly across said central axis at a cephalad end of said plate, said first sidewall further extends between said top and bottom surfaces of said plate; and
a second sidewall extending across said central axis at a caudal end of said plate between said lateral portions, wherein said second sidewall extends around a lobe centered on and extending caudally from said central portion along said central axis, and said second sidewall further extends between said top and bottom surfaces of said plate.

18. The plating system of claim 11, wherein said top surface is convexly curved around said medial-lateral axis from said bottom surface toward where said top defines said first thickness with said bottom surface.
